(19) United States
(12) Patent Application Publication
(10) Pub. No.: US 2024/0173141 A1
(43) Pub. Date: May 30, 2024

(54) KNEE-JOINT ENDOPROSTHESIS

(71) Applicant: Aesculap AG, Tuttlingen (DE)

(72) Inventor: Harry Kempf, Stetten (DE)

(21) Appl. No.: 18/519,330

(22) Filed: Nov. 27, 2023

(30) Foreign Application Priority Data
Nov. 29, 2022 (DE) 10 2022 212 768.4

(51) Int. Cl. A61F 2/38

(52) U.S. Cl. CPC A61F 2/389; A61F 2/3836; A61F 2002/30535

(57) ABSTRACT

A knee-joint endoprosthesis, which can be used for a knee-joint replacement operation, has a tibia component, a meniscus component and a screwed connection. The tibia component has a distal fastening portion and a proximal connecting portion. The distal fastening portion is configured for fastening to a proximal tibia. The meniscus component has a proximal sliding surface and a distal mating connecting portion. The proximal sliding surface is configured to articulate with a femoral joint component. The distal mating connecting portion is configured to form a joining connection with the proximal connecting portion of the tibia component. The screwed connection, which is configured for additional fixation of the joining connection, has a screw element inserted in a longitudinal bore of the meniscus component, and a mating screw element assigned to the tibia component. The screw element can be screwed to the mating screw element to form the screwed connection.

### CROSS-REFERENCE TO RELATED APPLICATIONS

This application claims priority under 35 U.S.C. § 119 to German Application No. 10 2022 212 768.4, filed on Nov. 29, 2022, the content of which is incorporated by reference herein in its entirety.

### FIELD

The disclosure relates to a knee-joint endoprosthesis, having a tibia component with a distal fastening portion, which is configured for fastening to a proximal tibia, and with a proximal connecting portion, and having a meniscus component with a proximal sliding surface, which is configured to articulate with a femoral joint component, and with a distal mating connecting portion, which is configured to form a joining connection with the proximal connecting portion of the tibia component.

### BACKGROUND

Knee-joint endoprostheses are well known in the field of orthopaedic surgery and serve as artificial replacements for natural knee joints. Conventional knee-joint endoprostheses have a tibia component and a meniscus component. The tibia component is configured for fastening to a proximal tibia of the patient and, to this end, has a distal fastening portion, which may for example be in the form of an intramedullary fastening shaft. The meniscus component has a proximal sliding surface for sliding articulation with a femoral joint component. The femoral joint component may be the natural distal femur, more specifically: its condyle surfaces, or a prosthetic femoral component. During the implantation of the knee-joint endoprosthesis, the meniscus component is connected to the tibia component, which is usually already fastened to the proximal tibia. To this end, the tibia component has a proximal connecting portion and the meniscus component has a complementary distal mating connecting portion. The connecting portion and the mating connecting portion are configured to form a joining connection, which is usually a plug-in, latching and/or snap-fit connection.

### SUMMARY

It is an object of the present disclosure to provide a knee-joint endoprosthesis which is of the type mentioned in the introduction and has improved properties in relation to the prior art.

This object is achieved in that a screwed connection is provided and configured for additional fixation of the joining connection, wherein the screwed connection has at least one screw element, which is inserted in a longitudinal bore of the meniscus component, and at least one mating screw element, which is assigned to the tibia component and to which the screw element can be screwed to form the screwed connection. The solution according to the disclosure has the effect of additional fixing of the joining connection. This counteracts inadvertent detachment of the joining connection. Inadvertent detachment of the joining connection in the implanted state of the knee-joint endoprosthesis adversely affects its function, leads to considerable injury to the patient and usually means that revision surgery is necessary. These disadvantages are overcome by the additional screwed connection according to the disclosure between the meniscus component and the tibia component. The screwed connection has the at least one screw element and the at least one mating screw element. The screw element and the mating screw element complement one another. The screw element is assigned to the meniscus component. The mating screw element is assigned to the tibia component. In one embodiment, the screw element is a screw and the mating screw element is a complementary threaded bore. The terms “screwed connection”, “screw element”, “mating screw element” and “screwed” are not to be interpreted as limiting in this respect. In a further embodiment, the screwed connection is in the form of a bayonet closure connection, wherein the screw element and the mating screw element are in the form of mutually complementary bayonet closure elements.

The longitudinal bore introduced in the meniscus component is configured to receive the screw element. The longitudinal bore is elongate axially along a bore axis which has at least one proximodistal direction component. In one embodiment, the bore axis moreover has an anteroposterior direction component. In a further embodiment, the bore axis alternatively or additionally has a mediolateral direction component. Expressed differently: The longitudinal bore is elongate in the broadest sense through the meniscus component from top to bottom, that is to say between a proximal top side and a distal bottom side of the meniscus component, wherein the longitudinal extent does not necessarily have to extend parallel to or even coaxially with a proximodistal longitudinal axis of the meniscus component. Preferably, the longitudinal bore is a through-bore. Preferably, the tibia component is made of a metallic material. Preferably, the meniscus component is made of a polymer material. Preferably, the screw element is made of a metallic material. In one embodiment, the mating screw element is introduced directly in the tibia component and/or formed by a portion of the tibia component. In a further embodiment, the mating screw element is a separate component and/or is formed by a portion of the separate component, wherein the separate component is connected to the tibia component at least in the implanted state.

The positional and directional designations used in this description relate to the body of a patient and in this respect are to be understood according to their conventional anatomical meaning. Therefore, “anterior” means front or at the front, “posterior” means rear or at the rear, “medial” means inner or on the inside, “lateral” means outer or on the outside, “proximal” means towards the centre of the body, and “distal” means away from the centre of the body. Furthermore, “proximodistal” means along, preferably parallel to, a proximal-distal axis, “anteroposterior” means along, preferably parallel to, an anterior-posterior axis, and “mediolateral” means along, preferably parallel to, a medial-lateral axis. Said axes are aligned orthogonally in relation to one another and may of course be understood in relation to X. Y and Z axes that are not linked to the anatomy of the patient. For example, the proximodistal axis may alternatively be denoted the Z axis. The mediolateral axis may be denoted the Y axis. The anteroposterior axis may be denoted the X axis. For the sake of better clarity and simplicity of the designations, said anatomical positional and directional designations are primarily used in the following text.

In one embodiment, the longitudinal bore is inclined anteriorly in the proximal direction relative to a proximodistal longitudinal axis of the meniscus component. This means that the bore axis of the longitudinal bore is elongate with a forward inclination. As a result, intraoperative access to the longitudinal bore is improved. The anterior inclination avoids a situation in which the femoral joint component conceals the longitudinal bore. A surgeon can therefore reach the longitudinal bore better in order, for example, to insert the screw element or to attach a tool shank for driving the screw element.

In one embodiment, the longitudinal bore has an insertion opening for inserting the screw element and/or for inserting a tool shank for driving the screw element. The insertion opening is arranged at one end of the longitudinal bore. Preferably, the insertion opening is arranged proximally and/or anteriorly on the meniscus component. The insertion opening in particular makes it possible to produce the screw connection even more easily.

In one embodiment, the insertion opening is arranged on an anterior front side of the meniscus component. This avoids a situation in which the femoral joint component covers the insertion opening. Therefore, the insertion opening is also easy for the operator to see and access during the knee joint replacement operation. As a result, the surgeon can insert the screw element and/or the tool shank into the insertion opening particularly easily.

In one embodiment, the insertion opening is arranged with a downward offset in the distal direction relative to an anterior edge of the sliding surface. This means the insertion opening can be seen and accessed particularly easily by the surgeon. The arrangement underneath the anterior edge of the sliding surface avoids a situation in which the insertion opening is concealed by the femoral joint component. This allows the surgeon to insert the tool shank for driving the screw element or the screw element itself into the insertion opening particularly easily.

In one embodiment, the screw element is retained captively in the longitudinal bore when the meniscus component is in a state separate from the tibia component. The captive retention ensures that the screw element does not inadvertently fall out of the longitudinal bore while the meniscus component is being attached to the tibia component. This allows particularly casy and reliable handling. Moreover, patient safety is increased. Preferably, at least one head portion of the screw element is retained captively in the longitudinal bore. Preferably, the head portion has a recessed drive geometry for receiving a complementary drive geometry of a tool shank. For the purpose of captive retention, the screw element is preferably retained radially and/or axially in the longitudinal bore. The captive retention of the screw element is preferably established already during the production of the knee-joint endoprosthesis. Therefore, it is also possible to say that the screw element is pre-mounted captively on the meniscus component.

In one embodiment, the longitudinal bore has a first bore portion, in which the screw element is captively retained, and a second bore portion, which has the insertion opening at one end and leads into the first bore portion at the other end. The screw element, preferably its head portion, is retained in the first bore portion in a radial and/or axial form fit. Preferably, the first bore portion to this end is dimensionally matched to the screw element, preferably its head portion. In this embodiment of the disclosure, the insertion opening is designed, preferably exclusively, for insertion of the said tool shank. The screw element is introduced and captively pre-mounted in the first bore portion preferably already during the production of the knee-joint endoprosthesis. Preferably, at one end the first bore portion leads into the second bore portion. At the other end, the first bore portion preferably has a mounting opening. The screw element is captively mounted preferably via the mounting opening. Preferably, the longitudinal bore has the insertion opening at one end and the mounting opening at the other end.

In one embodiment, a diameter of the insertion opening is smaller than a diameter of the first bore portion. This is a particularly preferred embodiment of the disclosure. The relatively reduced diameter of the insertion opening avoids tissue irritation and associated injuries to the patient in the use of the knee-joint endoprosthesis. The diameter of the insertion opening is reduced in relation to the diameter of the first bore portion and thus also to the diameter of the screw element, in particular its head portion. Therefore, it is also possible to say that the reduced diameter of the insertion opening leads to the screw element, in particular the head portion, being covered at least in certain portions. This covering counteracts said tissue irritation.

In one embodiment, the diameter of the insertion opening is less than 80%, preferably less than 60%, particularly preferably less than 50%, of the diameter of the first bore portion. The values denoted above have proven to be advantageous.

In one embodiment, the first bore portion is provided with an insert sleeve, in particular which is cast in the meniscus component and/or metallic, and in which the screw element is retained in a radial and axial form fit. The screw element, preferably its head portion, is retained in the insert sleeve in a force fit along its longitudinal axis at least in one direction, preferably in both directions, of the longitudinal axis. The insert sleeve allows easier captive mounting of the screw element on the meniscus component. If the insert sleeve is metallic, particularly reliable transmission of the forces of the screw connection from the screw element to the meniscus component can be achieved. This is advantageous in particular if the meniscus component is made of a polymer material.

In one embodiment, the mating screw element is a threaded bore. The threaded bore may be introduced directly in the tibia component. As an alternative, the threaded bore may be introduced in a separate component which is fixedly connected to the tibia component. The threaded bore is elongate coaxially with the longitudinal bore.

In one embodiment, the threaded bore is introduced in the fastening portion of the tibia component and/or is elongate coaxially with a proximodistal longitudinal axis of the tibia component. This allows particularly easy and cost-effective production.

In one embodiment, the threaded bore is introduced in a threaded sleeve inserted in the tibia component, in particular the fastening portion, and/or is inclined relative to a proximodistal longitudinal axis of the tibia component. This embodiment allows easier production, in particular if the longitudinal bore is anteriorly inclined in relation to the proximodistal longitudinal axis of the meniscus component and/or tibia component. An elongate threaded bore with an inclination coaxial therewith can be introduced directly in the fastening portion only with relatively high manufacturing outlay. The threaded sleeve allows simplification in this respect. Preferably, the fastening portion has a central bore, in which the threaded sleeve is fixedly inserted. The central bore is preferably parallel to a proximodistal longitudinal axis of the tibia component.

### BRIEF DESCRIPTION OF THE DRAWINGS

Further advantages and features of the disclosure will follow from the following description of preferred exemplary embodiments of the disclosure that are illustrated by means of the drawings.

FIG. 1 shows a perspective exploded view of one embodiment of a knee-joint endoprosthesis according to the disclosure.

FIG. 2 shows a schematic and slightly perspective view in longitudinal section of a knee-joint endoprosthesis according to FIG. 1.

FIG. 3 shows a schematic view in longitudinal section of a further embodiment of a knee-joint endoprosthesis according to the disclosure.

FIG. 4 shows a schematic perspective view of a further embodiment of a knee-joint endoprosthesis according to the disclosure.

FIG. 5 shows a schematic and slightly perspective view in longitudinal section of the knee-joint endoprosthesis according to FIG. 4, with individual components of the knee-joint endoprosthesis not yet being fully joined to one another.

FIG. 6 shows a further schematic and slightly perspective view in longitudinal section of the knee-joint endoprosthesis according to FIGS. 4 and 5 together with a schematically indicated tool shank.

### DETAILED DESCRIPTION

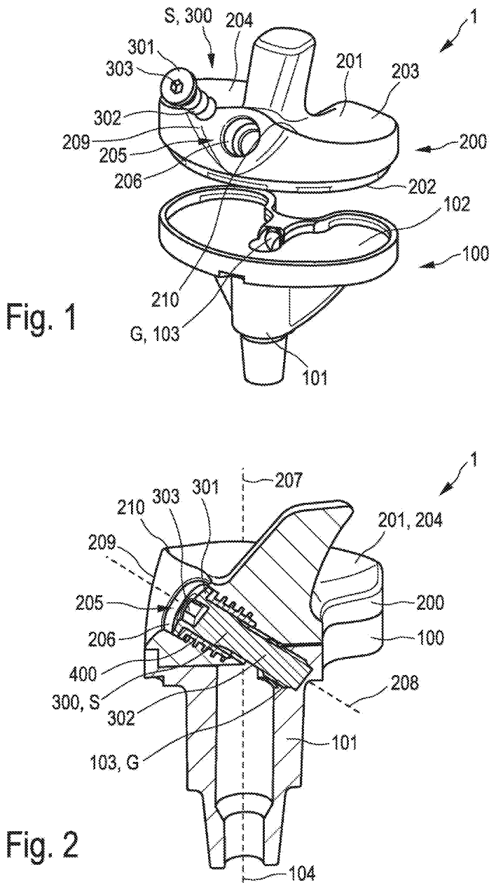

According to FIGS. 1 and 2, a knee-joint endoprosthesis 1 for use in a knee-joint replacement operation is provided and has a tibia component 100 and a meniscus component 200.

The tibia component 100 has a distal fastening portion 101 and a proximal connecting portion 102. The distal fastening portion 101 is configured for fastening to a proximal tibia. The distal fastening portion 101 can be screwed to or cemented in place in the proximal tibia in a way known to those skilled in the art. In the embodiment shown, the distal fastening portion is in the form of a proximodistal elongate fastening shaft. The proximal connecting portion serves for connection to the meniscus component 200. The tibia component 100 may also be referred to as tibial plateau.

The meniscus component 200 has a proximal sliding surface 201. The proximal sliding surface 201 is configured to articulate with a femoral joint component. The femoral joint component can be a prosthetic femur component, which may be a constituent part of the knee-joint endoprosthesis 1. As an alternative, and if the knee-joint endoprosthesis 1 only forms a partial replacement for a natural knee joint, the femoral joint component may be a natural distal femur. The proximal sliding surface 201 in the present case has two sliding-surface portions 203, 204. The sliding-surface portions 203, 204 are spaced apart from one another in the mediolateral direction and may also be referred to as lateral sliding-surface portion 203 and medial sliding-surface portion 204. The meniscus component 200 moreover has a distal mating connecting portion 202. The distal mating connecting portion 202 and the proximal connecting portion 102 complement one another and are configured to form a joining connection. In the present case, the mating connecting portion 202 can be latched to the connecting portion 102, with the result that the said joining connection is therefore a latching connection. In embodiments not shown in the drawings, the joining connection is a plug-in and/or clamping connection. Further details as to the form and function of the joining connection are not essential with respect to the present disclosure. Further explanations are therefore omitted in this respect.

For additional fixation of the joining connection between the connecting portion 102 and the mating connecting portion 202, a screwed connection is provided.

The screwed connection has a screw element 300 and a mating screw element 103. The screw element 300 is assigned to the meniscus component 200. The mating screw element 103 is assigned to the tibia component 100 and in the embodiment shown is formed by a portion of the tibia component 100 that is yet to be explained below. In further embodiments, the mating screw element may be formed by a separate component. To receive the screw element 300, the meniscus component 200 has a longitudinal bore 205, which is provided with an insertion opening 206 in the embodiment shown. In the embodiment shown, the insertion opening 206 serves for insertion of the screw element 300.

To attach the meniscus component 200 to the tibia component 100, firstly the mating connecting portion 202 is latched to the connecting portion 102. This forms the said joining connection. For additional fixation of the joining connection, the screw element 300 inserted in the longitudinal bore 205 of the meniscus component 200 is screwed to the mating screw element 103.

In the embodiment shown, the screw element 300 is a screw S and the mating screw element 103 is a threaded bore G. Such a design, however, is not mandatory. As an alternative, the screwed connection may be formed in the manner of a bayonet closure.

The meniscus component 200 is usually intraoperatively attached to the tibia component 100, which is already fastened to the tibia. To ensure easy accessibility of the screwed connection even during surgery, the longitudinal bore 205 is inclined anteriorly in the proximal direction relative to a proximodistal longitudinal axis 207 of the meniscus component 200. Expressed differently, a bore axis 208 of the longitudinal bore 205 is oriented obliquely in relation to the proximodistal longitudinal axis 207 such that the insertion opening 206 is arranged on an anterior front side 209 of the meniscus component 200.

In the fastened state shown by means of FIG. 2, the proximodistal longitudinal axis 207 is aligned coaxially with a proximodistal longitudinal axis 104 of the tibia component 100.

The sliding surface 201 has an anterior edge 210. The insertion opening 206 is arranged offset in the distal direction relative to the anterior edge 210. The arrangement of the insertion opening 206 on the anterior front side 209 and/or underneath the anterior edge 210 has the effect of particularly easy accessibility of the screw connection.

In the embodiment shown, the longitudinal bore 205 and/or its bore axis 208 is arranged in a centre longitudinal plane with a proximodistal and anteroposterior extent of the knee-joint endoprosthesis 1. In this case, the longitudinal bore 205 and/or the bore axis 208 have a forward inclination, in the present case by approximately 60°, from the proximodistal longitudinal axis 207. In further embodiments, the angle of inclination is for example between 10° and 80°.

In the embodiment shown, the mating screw element 103 in the form of the threaded bore G is introduced directly in the tibia component 100. It will be understood that the threaded bore G is aligned coaxially with the longitudinal bore 205. With reference to FIG. 2, the bore axis 208 denotes both the longitudinal axis of the longitudinal bore 205 and the longitudinal axis of the threaded bore G. In this respect, the statements made regarding the inclination of the longitudinal bore 205 also apply, mutatis mutandis, to the inclination of the threaded bore G and to the inclination of the screw element 300. In the embodiment shown, the threaded bore G is a through-bore and extends from the proximal connecting portion 102 distally and posteriorly through the tibia component 100, in particular through its fastening portion 101.

In all other respects, the screw S has a form which is known in principle to those skilled in the art, with a head portion 301, a shaft portion 302 and a drive geometry 303. The drive geometry 303 is recessed in the head portion 301 and can also be referred to as tool surface. The drive geometry 303 is configured to interact with a complementary drive geometry of a tool and/or of a tool shank. The shaft portion 302 is provided with a thread, not denoted in more detail, which has a complementary design to the threaded bore G and can be screwed to the latter. With reference to FIG. 2, the head portion 301 can be accessed through the insertion opening 206.

In the embodiment according to FIGS. 1 and 2, the knee-joint endoprosthesis 1 moreover has a compression sleeve 400, which is inserted in the meniscus component 200, more specifically its longitudinal bore 205. The compression sleeve 400 serves in particular to mechanically reinforce the longitudinal bore 205 and interacts directly with the screw element 300. The meniscus component 200 in the present case is manufactured from a polymer material. The compression sleeve 400 is made of a metallic material. It will be understood that the compression sleeve 400 is not mandatory. Accordingly, further embodiments do not have such a compression sleeve.

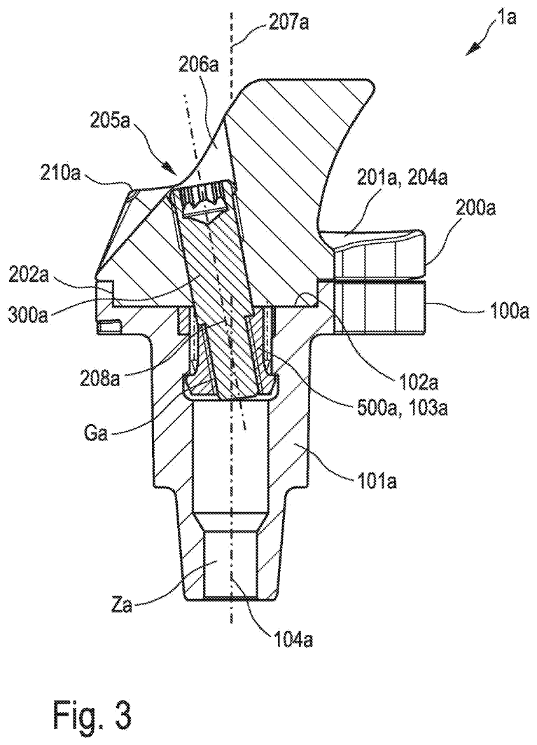

FIGS. 3 to 6 show further embodiments of knee-joint endoprostheses 1a, 1b according to the disclosure. To avoid repetitions, only essential differences of the knee-joint endoprostheses 1a, 1b with respect to the knee-joint endoprosthesis 1 according to FIGS. 1 to 2 will be explained in detail below. The disclosure relating to the knee-joint endoprosthesis 1 according to FIGS. 1 and 2 applies, mutatis mutandis, and, unless described otherwise, also to the knee-joint prostheses 1a, 1b.

The knee-joint endoprosthesis 1a according to FIG. 3 essentially differs from the knee-joint endoprosthesis 1 according to FIGS. 1 and 2 in that the threaded bore Ga is not introduced directly in the tibia component 100a. Instead, the threaded bore Ga is formed on a threaded sleeve 500a. The threaded sleeve 500a acts as a mating screw element 103a. The threaded sleeve 500a is fixedly connected to the tibia component 100a. To this end, the threaded sleeve 500a in the present case is introduced in and fixedly connected to a central bore Za of the fastening portion 101a. The central bore Za is oriented coaxially with the proximodistal longitudinal axis 104a of the tibia component 100a. By contrast, the threaded bore Ga is aligned obliquely in relation to the proximodistal longitudinal axis 104a. More specifically, the threaded bore Ga is anteriorly inclined in the manner already explained in connection with the knee-joint endoprosthesis 1 according to FIGS. 1 and 2. The threaded sleeve 500a in particular allows easier manufacture. Specifically, it is possible to omit the introduction of an inclined bore directly in the tibia component 100a.

Another difference in relation to the knee-joint endoprosthesis 1 according to FIGS. 1 and 2 is that the knee-joint endoprosthesis 1a does not have a compression sleeve 400.

The longitudinal bore 205a and/or the bore axis 208a moreover has a smaller angle of inclination. This is approximately 10°. Owing to the smaller anterior inclination, the insertion opening 206a—by contrast to the embodiment according to FIGS. 1 and 2—is not arranged underneath the anterior edge 210a of the sliding surface 201a.

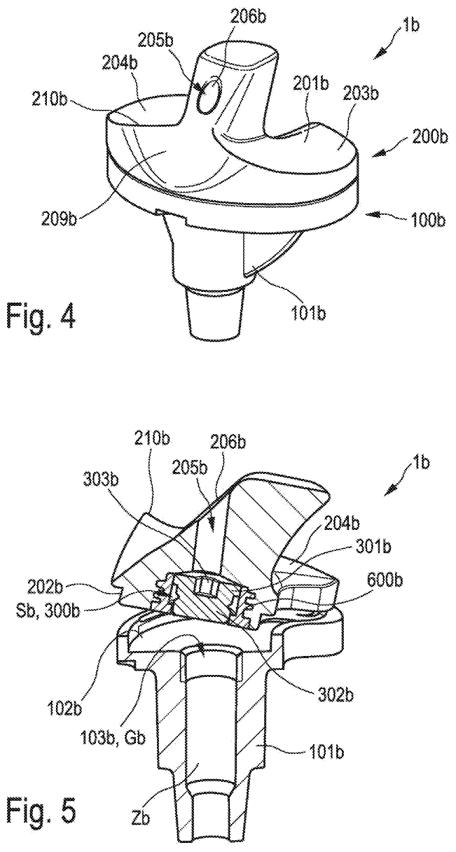

The knee-joint endoprosthesis 1b according to FIGS. 4 to 6 differs essentially in that the screw element 300b—when the meniscus component 200b is in a state separate from the tibia component 100b—is captively retained in the longitudinal bore 205b. This counteracts the screw element 300b inadvertently falling out of the longitudinal bore 205b. This allows easy and secure handling of the meniscus component 200 before and during attachment to the tibia component 100b.

For the purpose of captive retention, the screw element 300b is retained in the longitudinal bore 205b in a radial and axial form fit. The form fit in the axial direction permits limited movability of the screw element 300a in the present case.

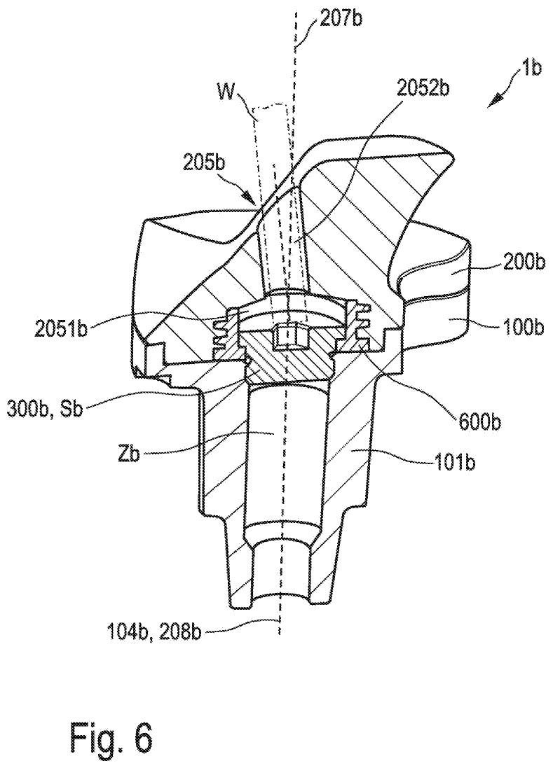

To attach the meniscus component 200b to the tibia component 100b, firstly the joining connection, already explained with regard to the embodiment according to FIGS. 1 and 2, between the connecting portion 102b and the mating connecting portion 202b is formed. After this, the screw element 300b is screwed to the mating screw portion 103b. To that end, a tool shank W, which is schematically indicated in FIG. 6, is inserted into the longitudinal bore 205b through the insertion opening 206b and brought into engagement with the drive geometry 303b. By rotating the tool shank W, a rotational movement and a torque are transmitted to the screw element 300b and the screw element 300b is therefore screwed to the mating screw element 103b.

In the embodiment shown, the longitudinal bore 205b has a first bore portion 2051b and a second bore portion 2052b (FIG. 6). The screw element 300b is captively retained in the first bore portion 2051b. The second bore portion 2052b has the insertion opening 206b at one end and leads into the first bore portion 2051b at the other end. In this case, the two bore portions 2051b, 2052b have different diameters. The diameter (without reference sign) of the second bore portion 2052b is smaller than the diameter (without reference sign) of the first bore portion 2051b. Therefore, the insertion opening 206b arranged at one end of the second bore portion 2052b also has a smaller diameter than the first bore portion 2051b. The diameter of the first bore portion 2051b is dimensionally matched to the screw element 300b that is to be received and is slightly larger than an outside diameter (without reference sign) of the head portion 301b of the screw element 300b. The reduced insertion opening relative to the diameter of the screw element 300b avoids irritation of the surrounding body tissue and thus associated injury to the patient.

In the embodiment shown, the diameter of the insertion opening 206b and/or of the second bore portion 2052b is approximately 50% of the diameter of the first bore portion 2051b.

To captively retain the screw element 300b in the longitudinal bore 205b, in the present case there is an insert sleeve 600b. The insert sleeve 600b forms the first bore portion 2051b. Expressed differently, it is also possible to say that the first bore portion 2051b is provided with the insert sleeve 600b. The insert sleeve 600b is made of a metallic material and is fixedly inserted in the meniscus component 200b. In the present case, the insert sleeve 600b is cast in the polymer meniscus component 200b. Such a form, however, is not mandatory. For example, the insert sleeve 600b may also be fixedly connected to the meniscus component 200b in another way known to those skilled in the art.

The screw element 300b is retained in a radial form fit in the insert sleeve 600b so as to be able to move axially to a limited extent. To this end, the insert sleeve 600b has a radial collar 601b, which acts as a distal stop for the head portion 301b of the screw element 300b. The radial collar 601b is arranged distally on the insert sleeve 600b. A length, not denoted in more detail, of the shaft portion 302b is dimensioned such that, its thread can be screwed to the mating screw element 103b with sufficient axial overlap in an end position—delimited in form-fitting fashion by the radial collar 601b—of the screw element 300b.

In the embodiment according to FIGS. 4 to 6, the mating screw element 103b is in turn in the form of a threaded bore Gb. The threaded bore Gb is introduced directly in the tibia component 100b and coaxially with its central bore Zb. The central bore Zb is elongate by virtue of the fastening portion 101b, is in the form of a through-bore and at one end—at its proximal end—has the threaded bore Gb. The threaded bore Gb—and thus also the screw element 300b—is coaxial with the proximodistal longitudinal axis 104b. Therefore, the bore axis 208b is likewise coaxial. This is the case at least in the region of the first bore portion 2051b. The second bore portion 2052b in the present case has slightly less of an anterior inclination (see FIG. 6). The anterior inclination in turn allows improved accessibility of the insertion opening 206b and easier insertion of the tool shank W for driving the screw element 300b. In an embodiment not shown in the drawings, the longitudinal bore does not have an anterior inclination and/or is not parallel to the proximodistal longitudinal axis of the meniscus component.

It will be understood that individual features of the knee-joint endoprostheses 1, 1a, 1b can be combined with one another to form further embodiments. For example, the knee-joint endoprostheses 1, 1a according to FIGS. 1, 2 and 3 can each be provided with captive retention of the screw element. Conversely, the knee-joint endoprosthesis 1b may be provided with a longitudinal bore with an anterior inclination (completely and not just in certain portions).

1. A knee-joint endoprosthesis comprising:
a tibia component comprising a distal fastening portion and a proximal connecting portion, the distal fastening portion configured for fastening to a proximal tibia;
a meniscus component comprising a proximal sliding surface and a distal mating connecting portion, the proximal sliding surface configured to articulate with a femoral joint component, and the distal mating connecting portion configured to form a joining connection with the proximal connecting portion of the tibia component; and
a screwed connection configured for additional fixation of the joining connection,
the screwed connection comprising at least one screw element and at least one mating screw element,
the at least one screw element being inserted in a longitudinal bore of the meniscus component,
the at least one mating screw element being assigned to the tibia component, and
the screw element configured to be screwed to the at least one mating screw element to form the screwed connection.

2. The knee-joint endoprosthesis according to claim 1, wherein the longitudinal bore is inclined anteriorly in a proximal direction relative to a proximodistal longitudinal axis of the meniscus component.

3. The knee-joint endoprosthesis according to claim 1, wherein the longitudinal bore has an insertion opening for inserting the screw element and/or for inserting a tool shank for driving the screw element.

4. The knee-joint endoprosthesis according to claim 3, wherein the insertion opening is arranged on an anterior front side of the meniscus component.

5. The knee-joint endoprosthesis according to claim 3, wherein the insertion opening is arranged with a downward offset in a distal direction relative to an anterior edge of the sliding surface.

6. The knee-joint endoprosthesis according to claim 1, wherein the screw element is retained captively in the longitudinal bore when the meniscus component is in a state separate from the tibia component.

7. The knee-joint endoprosthesis according to claim 6, wherein the longitudinal bore has a first bore portion, in which the screw element is captively retained, and a second bore portion, which has the insertion opening at a first end and leads into the first bore portion at a second end.

8. The knee-joint endoprosthesis according to claim 7, wherein a diameter of the insertion opening is smaller than a diameter of the first bore portion.

9. The knee-joint endoprosthesis according to claim 8, wherein the diameter of the insertion opening is less than 80%.

10. The knee-joint endoprosthesis according to claim 7, wherein the first bore portion is provided with an insert sleeve, in which the screw element is retained in a radial and axial form fit.

11. The knee-joint endoprosthesis according to claim 1, wherein the mating screw element is a threaded bore.

12. The knee-joint endoprosthesis according to claim 11, wherein the threaded bore is introduced in the fastening portion of the tibia component and/or is elongate coaxially with a proximodistal longitudinal axis of the tibia component.

13. The knee-joint endoprosthesis according to claim 11, wherein the threaded bore is introduced in a threaded sleeve that is at least one of:
inserted in the tibia component, and
inclined relative to a proximodistal longitudinal axis of the tibia component.
